(19) United States
(12) Patent Application Publication
(10) Pub. No.: US 2022/0133705 A1
(43) Pub. Date: May 5, 2022

(54) Composition for increasing skin thickness

(71) Applicant: PHYSIOMEFIT, LLC, Stuart, FL (US)

(72) Inventor: Paul J. Roubal, Stuart, FL (US)

(21) Appl. No.: 17/519,771

(22) Filed: Nov. 5, 2021

Related U.S. Application Data
(60) Provisional application No. 63/110,120, filed on Nov. 5, 2020.

(51) Int. Cl. A61K 31/455; A61K 31/07; A61K 31/375; A61K 31/355; A61K 31/198; A61K 31/4172; A61K 31/405; A61K 9/00; A61P 17/00

(52) U.S. Cl. CPC A61K 31/455; A61K 31/07; A61K 31/375; A61K 31/355; A61P 17/00; A61K 31/4172; A61K 31/405; A61K 9/0014; A61K 31/198

(57) ABSTRACT

A composition and method to increase skin thickness. The composition, preferably a topical lotion, may comprise skin thickening agents and a delivery agent, which in combination increases skin thickness, and may help reduce the possibility of injuries related to individuals suffering from thin or thinning skin, such as bruising or skin tears.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/110,120, entitled “COMPOSITION FOR INCREASING SKIN THICKNESS”, filed Nov. 5, 2020. The contents of the above referenced applications are incorporated herein by reference in its entirety.

### FIELD OF THE INVENTION

The present invention relates to a medical composition; to a composition for use in resolving issues related to thinning skin; and more particularly, to a composition for topical application to an individual which increases skin thickness.

### BACKGROUND OF THE INVENTION

Often overlooked as a critical component of an individual's health is skin. Being the largest organ in the human body, it generally covers the entire body and serves as a means of protection, regulation and sensation. Healthy skin protects humans from microbes and the elements, helps to regulate body temperature, and permits the sensations of touch, heat, and cold. It is made of three layers, the epidermis, the dermis, and the hypodermis.

The epidermis, being the top layer of the skin, functions to protect the underlying skin layers from the outside environment and contains cells that make keratin. The epidermis is also responsible for the skin color, containing cells that produce melanin. The epidermis also contains cells that allow humans to feel the sensation of touch and provide the body with immunity against pathogens, such as viruses and bacteria. The middle layer, the dermis, contains cells that give skin strength, support, and flexibility. The fibroblast cells of the dermis are responsible for manufacturing collagen, elastin, heparan sulfate, and hyaluronic acids, which all play a role in the health and appearance of the skin. As a person ages, the cells in the dermis lose their strength and flexibility, causing skin issues. This is a result of the decline in functional fibroblasts and the production of collagen, heparan sulfate, and hyaluronic acids slowing down. As a result, as it is not uncommon for older individuals to suffer from thinning skin, wrinkles, skin bruising, and skin sagging. The dermis may also contain sensory receptors that allow the body to receive outside environmental stimulations. These sensory cells further allow individuals to feel pressure, pain, and temperature. The bottom layer, the hypodermis, contains adipose tissue that insulates the body and helps it conserve heat.

As an individual gets older, skin thinning, particularly on one's arms and legs, can be a significant problem. Skin thinning often leads to secondary issues, such as bruising and skin tears. Accordingly, there is a need for a composition that addresses the issues related to thinning of the skin, reduces associated injuries, and reduces medicals costs associated with thinning of the skin.

### SUMMARY OF THE INVENTION

The present invention relates to a composition and method to increase skin thickness. The present invention relates to a composition, preferably a topical lotion, comprising skin thickening agents and a delivery agent. The composition may include one or more of: one or more essential amino acids, and one or more vitamins, in any combination. The composition may also include sodium.

Accordingly, it is an objective of the invention to provide a composition for use with individuals suffering from thin/thinning skin.

It is a further objective of the invention to provide a method for treating or preventing thin skin.

It is yet another objective of the invention to provide a composition that thickens skin.

It is a still further objective of the invention to provide a composition that thickens skin and reduces injuries related to thin skin.

It is a further objective of the invention to provide a method of thickening skin.

It is yet another objective of the invention to provide a method that thickens skin and reduces injuries related to thin skin.

It is a still further objective of the invention to provide a composition that increases skin thickness by increasing collagen in the skin.

It is a further objective of the invention to provide a method that increases skin thickness by increasing collagen in the skin.

It is a still further objective of the invention to provide a composition having, in combination, skin thickening agents and a transport agent that increases skin thickness by increasing collagen in the skin.

It is a still further objective of the invention to provide a composition for promoting increases in skin thickness having, in combination, vitamins and amino acids and a transport agent for applying to an individual in need thereof.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### DETAILED DESCRIPTION OF THE INVENTION

While the present invention is susceptible of embodiment in various forms, there is described a presently preferred, albeit not limiting, embodiment with the understanding that the present disclosure is to be considered an exemplification of the present invention and is not intended to limit the invention to the specific embodiments illustrated.

The present invention provides a composition for increasing skin thickness in an individual. The present invention further provides a method of increasing skin thickness of an individual in need thereof. The composition may comprise skin thickening agents (which may also be defined as the active ingredients) and a transport agent which, in combination, increases skin thickness, and may help reduce the possibility of injuries related to individuals suffering from thin or thinning skin, such as bruising or skin tears.

As used herein, the term “effective amount” includes a sufficient amount of the skin thickening composition, when referring to the skin thickening composition in general, to provide the desired local or system effect, preferably without adverse side effects (i.e. unwanted or adverse effects of active ingredients or the composition as a whole), or to at least partially attain the desired effect. When referring to the individual component(s) of the skin thickening composition, “effective amount” may be defined as a sufficient amount of the individual component(s) to provide its intended or desired effect.

As used herein, the term “active ingredient” or “physiologically active ingredient” may be defined as ingredients, agents, compounds, individually or in combination, which exert a physiological effect, such as increase skin thickness, increase collagen levels, or reduce injures that result from thinning skin, such as bruising or skin tears.

As used herein, the term “about” can be defined as any value that is effective for its function, or a value which is 10%-35% above or below the stated concentration or amount.

The skin thickening composition may include effective amounts of one or more (in any combination) vitamins, such as but not limited to, Vitamin A, Vitamin B3 (Niacin), Vitamin C, and Vitamin E.

The skin thickening composition may include effective amounts of one or more amino (in any combination) acids, such as, but not limited to, Alanine, Cysteine, Aspartic Acid, Glutamic Acid, Phenylalanine, glycine, Histidine, Isoleucine, Lysine, Leucine, Methionine, Asparagine, Proline, Glutamine, Arginine, Serine, Threonine, Valine, Tryptophan, and Tyrosine. The amino acid may be in the L-stereoisomer form or the D-stereoisomer form. The amino acids may be supplied in a purified form or as part of a protein composition that can be broken to form or provide the amino acid. The amino acids may be supplied in powder form and mixed with water or other solvents or in an oil. The use of the term “amino acids” may also include amino acid derivatives, either naturally occurring or man-made, such as for example, β-Alanine, or amino acid metabolites, such the proline metabolite, hydroxyproline.

The skin thickening composition may include sodium.

The skin thickening composition may include an effective amount of a delivery agent. The delivery agent may be a transdermal vehicle that transports or allows systemic absorption of the composition through the skin. The delivery agent may be a transdermal base, such as LIPODERM, pluronic lecithin organogel (PLO, vehicle consisting of water, pluronic acid, and lecithin), or HRT transfer agent (Hormone Replacement Therapy transfer agent, [LIPODERM, transdermal base with liposomal components, or ARTEVIS, hydrogel with skin permeation enhancers and polymer network]). The delivery agent may be added in any effective amount, such as, but not limited to between 30% and 70% of the skin thickening composition.

The skin thickening composition may include one or more non-physiologically active ingredients. As used herein, the use of the term “non-physiologically active ingredients” may be defined as ingredients, agents, compounds that may be admixed with the active ingredients, but do not function to provide or exert a physiological effect. The one or more (in any combination) non-physiologically active ingredients may include effective amounts of solvents, solubilizers, stiffening agents, surfactants, agents to prevent passage to lower skin layers, emulsifying agents, stabilizing agents, dispersing agents, thickening agents, coloring agents, preservatives (which may include anti-microbial agents, antifungal agents), anti-oxidants, emollients, humectants, skin protectants, viscosity stabilizers, buffers, chelating agents, other non-physiologically active ingredients known to one of skill in the art, or combinations thereof.

In one embodiment, the skin thickening composition may be a topical/transdermal lotion.

In one embodiment, the skin thickening composition may be a topical/transdermal cream.

In one embodiment, the skin thickening composition may be a topical/transdermal gel.

In one embodiment, the skin thickening composition may be a topical/transdermal ointment.

In one embodiment, the skin thickening composition may be in the form of a gel or liquid.

The skin thickening composition is designed to increase skin thickness of individuals, and may comprise, preferably in combination, skin thickening agents (which may also be defined as the active ingredients), a transport agent, and, optionally, one or more non-active ingredients/agents.

The following are illustrative examples the composition:

Skin thickening composition: Example 1A:

| Component | Concentration/Amount/Range |
|---|---|
| Skin thickening agent(s) | Effective amounts |
| Delivery agent(s) | Effective amounts |

Skin thickening composition: Example 1B:

| Component | Concentration/Amount/Range |
|---|---|
| Skin thickening agent(s) | Effective amounts |
| Delivery agent(s) | Effective amounts |
| Sodium | Effective amounts |

Skin thickening composition: Example 2A

| Component | Concentration/Amount/Range |
|---|---|
| Skin thickening agent(s) | Effective amounts |
| Delivery agent(s) | Effective amounts |
| Non-physiologically active ingredients | Effective amounts |

Skin thickening composition: Example 2B

| Component | Concentration/Amount/Range |
|---|---|
| Skin thickening agent(s) | Effective amounts |
| Delivery agent(s) | Effective amounts |
| Sodium | Effective amounts |
| Non-physiologically active ingredients | Effective amounts |

Skin thickening composition: Example 3A

| Component | Concentration/Amount/Range |
|---|---|
| One or more vitamins | Effective amounts |
| One or more amino acids | Effective amounts |
| Transport molecule/agent | Effective amounts |
| Non-physiologically active ingredients | Optional/effective amounts |

Skin thickening composition: Example 3B

| Component | Concentration/Amount/Range |
|---|---|
| One or more vitamins | Effective amounts |
| One or more amino acids | Effective amounts |
| Sodium | Effective amounts |
| Transport molecule/agent | Effective amounts |
| Non-physiologically active ingredients | Optional/effective amounts |

Skin thickening composition: Example 4A

| Component | Concentration/Amount/Range (grams) |
|---|---|
| Vitamin A | About 0.01 g to about 0.1 |
| Vitamin C | About 15 g to about 60 g |
| Vitamin E | About 2.25 g to about 10 g |
| Niacin (Vitamin B-3) | About 2.25 g to about 10 g |
| L-Leucine | About 10 g to about 40 g |
| L-Valine | About 10 g to about 40 g |
| L-Isoleucine | About 10 g to about 40 g |
| L-Lysine | About 10 g to about 40 g |
| L-Phenylalanine | About 10 g to about 40 g |
| L-Threonine | About 10 g to about 40 g |
| L-Methionine | About 10 g to about 40 g |
| L-Tryptophan | About 10 g to about 40 g |
| L-Alanine | About 20 g to about 100 g |
| L-Arginine | About 15 g to about 70 g |
| L-Aspartic Acid | About 220 to about 900 g |
| L-Cysteine | About 15 g to about 60 g |
| L-Glutamic Acid | About 20 g to about 90 g |
| L-Glycine | About 50 g to about 200 g |
| L-Histidine | About 15 g to about 60 g |
| L-Isoleucine | About 3 g to about 20 g |
| L-Proline | About 35 g to about 140 g |
| L-Serine | About 5 g to about 30 |
| L-Threonine | About 5 g to about 20 |
| L-Tyrosine | About 1 g to about 10 g |
| Sodium | About 50 g to about 225 g |
| Delivery Agent:HRT transfer | about 25% to about 85% |
| agent | |
| Non-physiologically active | Optional/effective amounts |
| ingredients | |

Skin thickening composition: Example 4B

| Component | Concentration/Amount/Range (grams) | |
|---|---|---|
| Vitamin A | 0.05 | g |
| Vitamin C | 30 | g |
| Vitamin E | 4.5 | g |
| Niacin (Vitamin B-3) | 4.5 | g |
| L-Leucine | 18 | g |
| L-Valine | 18 | g |
| L-Isoleucine | 18 | g |
| L-Lysine | 18 | g |
| L-Phenylalanine | 18 | g |
| L-Threonine | 18 | g |
| L-Methionine | 19 | g |
| L-Tryptophan | 18 | g |
| L-Alanine | 4 0 | g |
| L-Arginine | 35 | g |
| L-Aspartic Acid | 438 | g |
| L-Cysteine | 2 8 | g |
| L-Glutamic Acid | 45 | g |
| L-Glycine | 100 | g |
| L-Histidine | 30 | g |
| L-Isoleucine | 7 | g |
| L-Proline | 68 | g |
| L-Serine | 14 | g |
| L-Threonine | 9 | g |
| L-Tyrosine | 3 | g |
| Sodium | 110 | g |

| | |
|---|---|
| Delivery Agent:HRT transfer | Effective amounts |
| agent | |
| Non-physiologically active | Optional/effective amounts |
| ingredients | |

Skin thickening composition: Example 5A

| Component | Concentration/Amount/Range (grams) |
|---|---|
| Vitamin A | about 0.01 g to about 0.1 g |
| Vitamin C | about 10 g to about 60 g |
| Vitamin E | about 2.25 g to about 9 g |
| Niacin (Vitamin B-3) | about 2.25 g to about 9 g |
| L-Alanine | about 20 g to about 100 g |
| L-Arginine | about 20 g to about 100 g |
| L-Aspartic Acid | about 10 g to about 75 g |
| L-Glutamic Acid | about 35 g to about 140 g |
| L-Glycine | about 55 g to about 225 g |
| L-Histidine | about 1 g to about 10 g |
| L-Hydroxylysine | about 30 g to about 125 g |
| L-Isoleucine | about 5 g to about 25 g |
| L-Leucine | about 5 g to about 50 g |
| L-Lysine | about 10 g to about 50 g |
| L-Methionine | about 1 g to about 10 g |
| L-Phenylalanine | about 5 g to about 25 g |
| L-Proline | about 30 g to about 125 g |
| L-Serine | about 10 g to about 50 g |
| L-Threonine | about 5 g to about 20 g |
| L-Tyrosine | about 1 g to about 6 g |
| L-Valine | about 5 g to about 40 g |
| L-Hydroxyproline | about 30 g to about 125 g |
| Sodium | about 50 g to about 225 g |
| Delivery Agent:HRT transfer | about 25% to about 85% |
| agent | |
| Non-physiologically active | Optional/effective amounts |
| ingredients | |

Skin thickening composition: Example 5B

| Component | Concentration/Amount (grams) | |
|---|---|---|
| Vitamin A | 0.05 | g |
| Vitamin C | 30 | g |
| Vitamin E | 4.5 | g |
| Niacin (Vitamin B-3) | 4.5 | g |
| L-Alanine | 44 | g |
| L-Arginine | 45 | g |
| L-Aspartic Acid | 3 6 | g |
| L-Glutamic Acid | 67 | g |
| L-Glycine | 111 | g |
| L-Histidine | 4 | g |
| L-Hydroxylysine | 62 | g |
| L-Isoleucine | 8 | g |
| L-Leucine | 16 | g |
| L-Lysine | 18 | g |
| L-Methionine | 3 | g |
| L-Phenylalanine | 11 | g |
| L-Proline | 62 | g |
| L-Serine | 18 | g |
| L-Threonine | 10 | g |
| L-Tyrosine | 3 | g |
| L-Valine | 13 | g |
| L-Hydroxyproline | 62 | g |
| Sodium | 110 | g |

| | |
|---|---|
| Delivery Agent:HRT transfer | Effective amounts |
| agent | |
| Non-physiologically active | Optional/effective amounts |
| ingredients | |

In use, the method of increasing skin thickness of an individual in need thereof, may comprise applying to an individual's skin a topical or transdermal composition, such as any composition described in Example 1-5B. The composition may be applied daily, such as twice a day or as needed.

Results of Application of a topical or transdermal composition as described in Examples 1-5B.

The composition was applied to the same location, for example, on the dorsal forearms, elbow to the wrist, on an individual male, over 50 years old, daily 2 time a day, over a predetermined time period. Skin thickness measurements were undertaken using high frequency ultrasound. Measurements were taken at several locations. Preferably, the composition was applied after a bath or shower when the skin was wet to aid in absorption through the skin.

A. First Application applied to the left and right arm: Date Applied: Nov. 24, 2020

Initial skin measurements:

Right Prox: 0.18 cm

Right Mid: 0.19 cm

Right Distal 0.27 cm

Right Average: 0.213 cm

Left Prox: 0.23 cm

Left Mid: 0.21 cm

Left Distal: 0.23 cm

Left Avg: 0.22

Overall: 0.2165 cm

B. Second measurement date: Feb. 1, 2021

Right Prox; 0.25 cm

Right Mid: 0.23 cm

Right Distal: 0.21 cm

Right Average: 0.23 cm

Left Prox: 0.25 cm

Left Mid: 0.27

Left Distal: 0.27 cm

Left Average: 0.253 cm

Overall Average: 0.2475 cm

C. Third measurement date: May 26, 2021

Right Prox: 0.35 cm

Right Mid: 0.31 cm

Right Distal: 0.36 cm

Right average: 0.34 cm

Left Prox: 0.41 cm

Left Mid: 0.37 CM

Left Distal: 0.39 cm

Average Left: 0.39 cm

Overall skin thickness to date of third measurement: 0.365 cm

Change of thickness: 0.1485 cm, representing 65% increase in thickness of skin within 6 months.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A composition for increasing skin thickness in an individual comprising:
an effective amount of one or more skin thickening agents; and
an effective amount of a delivery agent.

2. The composition for increasing skin thickness in an individual according to claim 1, wherein said composition is a topical/transdermal lotion.

3. The composition for increasing skin thickness in an individual according to claim 1, wherein said composition is a topical/transdermal cream.

4. The composition for increasing skin thickness in an individual according to claim 1, wherein said composition is a topical/transdermal gel.

5. The composition for increasing skin thickness in an individual according to claim 1, wherein said composition is a topical/transdermal ointment.

6. The composition for increasing skin thickness in an individual according to claim 1, wherein said delivery agent is a transdermal vehicle that transports or allows systemic absorption of the composition through the skin

7. The composition for increasing skin thickness in an individual according to claim 1, wherein said composition includes one or more non-physiologically active ingredients.

8. The composition for increasing skin thickness in an individual according to claim 7, wherein said one or more non-physiologically active ingredients includes solvents, solubilizers, stiffening agents, surfactants, agents to prevent passage to lower skin layers, emulsifying agents, stabilizing agents, dispersing agents, thickening agents, coloring agents, preservatives, anti-oxidants, emollients, humectants, skin protectants, viscosity stabilizers, buffers, chelating agents, or combinations thereof.

9. The composition for increasing skin thickness in an individual according to claim 1, wherein said one or more skin thickening agents includes one or more vitamins.

10. The composition for increasing skin thickness in an individual according to claim 9, wherein said one or more vitamins includes effective amounts of Vitamin A, Vitamin B3 (Niacin), Vitamin C, Vitamin E, or combinations thereof.

11. The composition for increasing skin thickness in an individual according to claim 1, wherein said one or more skin thickening agents includes one or more one or more amino acids.

12. The composition for increasing skin thickness in an individual according to claim 11, wherein said one or more one or more amino acids includes Alanine, Cysteine, Aspartic Acid, Glutamic Acid, Phenylalanine, glycine, Histidine, Isoleucine, Lysine, Leucine, Methionine, Asparagine, Proline, Glutamine, Arginine, Serine, Threonine, Valine, Tryptophan, Tyrosine, or combinations thereof.

13. The composition for increasing skin thickness in an individual according to claim 1, wherein said composition further includes sodium.

14. A composition for increasing skin thickness in an individual comprising:
one or more vitamins at a concentration of between about 0.01 grams and about 10 grams;
one or more amino acids at a concentration of between about 10 grams and 900 grams; and
a delivery agent at a concentration of about 25% to about 85%.

15. The composition for increasing skin thickness in an individual according to claim 14, wherein said composition includes effective amounts of one or more non-physiologically active ingredients.

16. The composition for increasing skin thickness in an individual according to claim X, wherein said one or more non-physiologically active ingredients includes solvents, solubilizers, stiffening agents, surfactants, agents to prevent passage to lower skin layers, emulsifying agents, stabilizing agents, dispersing agents, thickening agents, coloring agents, preservatives, anti-oxidants, emollients, humectants, skin protectants, viscosity stabilizers, buffers, chelating agents, or combinations thereof.

17. The composition for increasing skin thickness in an individual according to claim 14, wherein said composition further includes sodium.

18. A method for increasing skin thickness in an individual comprising:
applying a composition comprising one or more vitamins at a concentration of between about 0.01 grams and about 10 grams; one or more amino acids at a concentration of between about 10 grams and 900 grams; and a delivery agent at a concentration of about 25% to about 85%, to a treatment site;
wherein application of said composition to said treatment site results in an increase in skin thickness.

19. The method for increasing skin thickness in an individual according to claim 18, wherein said composition further includes sodium at a concentration of about 50 grams to about 225 grams.

20. The method for increasing skin thickness in an individual according to claim 18, wherein said composition includes effective amounts of one or more non-physiologically active ingredients.
